(19) United States
(12) Patent Application Publication
(10) Pub. No.: US 2022/0096263 A1
(43) Pub. Date: Mar. 31, 2022

(54) EXTERNAL URINE BAGS AND METHODS OF MAKING AND USING THE SAME

(71) Applicant: Sabry Gabriel, Lizella, GA (US)

(72) Inventor: Sabry Gabriel, Lizella, GA (US)

(21) Appl. No.: 17/488,919

(22) Filed: Sep. 29, 2021

Related U.S. Application Data
(60) Provisional application No. 63/085,227, filed on Sep. 30, 2020.

(51) Int. Cl. A61F 5/44; A61B 10/00; A61F 5/453; B29D 22/00

(52) U.S. Cl. CPC A61F 5/4407; A61B 10/007; B29D 22/003; A61F 5/453; A61F 5/4405

(57) ABSTRACT

Urine bags and kits containing a urine bag are disclosed. Methods of making and using urine bags are also disclosed.

### CROSS-REFERENCE TO RELATED APPLICATIONS

This patent application claims the benefit of priority to U.S. Provisional Patent Application Ser. No. 63/085,227 filed on Sep. 30, 2020 and entitled “EXTERNAL URINE BAGS AND METHODS OF MAKING AND USING THE SAME,” the subject matter of which is hereby incorporated by reference in its entirety.

### FIELD OF THE INVENTION

The present invention relates generally to external urine bags. The present invention further relates to methods of making and using external urine bags.

### BACKGROUND OF THE INVENTION

Indwelling urinary catheters are catheters inserted through the penile urethra into the urinary bladder. They are frequently the cause for urinary tract infections (UTI) or catheter associated urinary tract infections (CAUTI).

Efforts continue to further develop useful alternatives to indwelling catheters.

### SUMMARY OF THE INVENTION

The object of this invention is to provide a urine bag that provides an alternative to indwelling catheters. The disclosed urine bags may be adhesively attached to a user and sized so as to enclose the user's penis. The disclosed urine bags enable urine sample collection over an extended period of time (e.g., over a 24-hour period and up to one week).

In one exemplary embodiment of the present invention, the urine bag comprises a urine bag comprising: a front film having a front film outer perimeter, and a rear film having a rear film outer perimeter, the rear film outer perimeter being bonded to the front film outer perimeter so as to form a urine collection chamber between the front film and the rear film; an inner front film having an inner front film outer perimeter, and an inner rear film having an inner rear film outer perimeter, the inner front film outer perimeter being bonded to the inner rear film outer perimeter so as to form a penis-containing chamber between the front film and the rear film; an inlet within an upper half R_{UH} of (i) the rear film and (ii) the inner rear film and positioned a distance d_{IE} from the rear film outer perimeter, the inlet having an inlet diameter D_{I} of at least 20.0 millimeters (mm), more typically, from about 40.0 mm to about 60 mm, said inlet providing access to the penis-containing chamber; an outlet extending along a lower edge portion of the front film outer perimeter and the rear film outer perimeter, the outlet having an outlet diameter D_{O} of less than about 20.0 mm, said outlet providing an outlet for the urine collection chamber; and a fluid flow regulator that provides one-way fluid flow from the penis-containing chamber into the urine collection chamber. The urine bag may further comprise a number of additional components such as: an outlet tubular member extending from the outlet to a position below the lower edge portion, the outlet tubular member providing fluid communication between the urine collection chamber and an exit of the outlet tubular member; and an adhesive component that enables the urine bag to be adhesively attached to a user so as to encircle and contain the user's penis within the penis-containing chamber.

The present invention is further directed to methods of making urine bags. In one exemplary embodiment of the present invention, the method of making a urine bag comprises: bonding a front film having a front film outer perimeter to a rear film having a rear film outer perimeter so as to form a urine collection chamber between the front film and the rear film; bonding an inner front film having an inner front film outer perimeter to an inner rear film having an inner rear film outer perimeter so as to form a penis-containing chamber between the front film and the rear film; forming an inlet within an upper half R_{UH} of (i) the rear film and (ii) the inner rear film and positioned a distance d_{IE} from the rear film outer perimeter, the inlet having an inlet diameter D_{I} of at least 20.0 millimeters (mm), and providing access to the penis-containing chamber; forming an outlet extending along a lower edge portion of the front film outer perimeter and the rear film outer perimeter, the outlet having an outlet diameter D_{O} of less than about 20.0 mm, and providing an outlet for the urine collection volume; and positioning a fluid flow regulator between the penis-containing chamber and the urine collection chamber so as to provide one-way fluid flow from the penis-containing chamber into the urine collection chamber. In some embodiments, one or more of the bonding steps and the positioning steps may be performed sequentially or simultaneously.

In some embodiments, the methods of making urine bags may further comprise one or more of the following steps: providing an outlet tubular member extending from the outlet to a position below the lower edge portion of the urine bag, the outlet tubular member providing fluid communication between the urine collection chamber and an exit of the outlet tubular member; and providing an adhesive component that enables the urine bag to be adhesively attached to a user so as to encircle and contain the user's penis within the penis-containing chamber; cutting the inlet within the upper half of the rear films; and connecting the outlet tubular member to the front film and the rear film.

The present invention is even further directed to methods of using urine bags. In one exemplary embodiment of the present invention, the method of using a urine bag comprises adhesively attaching any one of the herein-described urine bags to a user so as to encircle and contain the user's penis within the penis-containing chamber of the urine bag; and collecting urine from the user within the urine collection chamber of the urine bag.

Other features and advantages of the present invention will become apparent after a review of the following detailed description of the disclosed embodiments and the appended claims.

### BRIEF DESCRIPTION OF THE FIGURES

The present invention is further described with reference to the appended figures, wherein:

FIG. 1 depicts a front view of an exemplary urine bag of the present invention;

FIG. 2 depicts a rear view of the exemplary urine bag shown in FIG. 1;

FIG. 3 depicts a rear view of an exemplary adhesive component suitable for use with the urine bag of the present invention;

FIG. 4 depicts a front view of the exemplary adhesive component shown in FIG. 3;

FIG. 5 depicts a rear view of another exemplary adhesive component suitable for use with the urine bag of the present invention;

FIG. 6 depicts a cross-sectional view of the exemplary adhesive component shown in FIG. 5 as viewed along line 6-6 shown in FIG. 5;

FIG. 7 depicts a cross-sectional view of the exemplary urine bag shown in FIG. 2 as viewed along line 7-7 shown in FIG. 2;

FIG. 8 depicts a cross-sectional view of the exemplary urine bag shown in FIG. 2 as viewed along line 8-8 shown in FIG. 2; See corrected figures.

FIG. 9 depicts a cross-sectional view of the exemplary urine bag shown in FIG. 2 as viewed along line 9-9 shown in FIG. 2;

FIG. 10 depicts a cross-sectional view of the exemplary urine bag shown in FIG. 2 as viewed along line 10-10 shown in FIG. 2;

FIG. 11 depicts a cross-sectional view of the exemplary urine bag shown in FIG. 2 as viewed along line 11-11 shown in FIG. 2;

FIG. 12 depicts a view of an exemplary valve suitable for use with the urine bag of the present invention;

FIG. 13A depicts a cross-sectional view of the exemplary valve shown in FIG. 12 as viewed along line 13-13 shown in FIG. 12, the exemplary valve being in a closed position;

FIG. 13B depicts a cross-sectional view of the exemplary valve shown in FIG. 12 as viewed along line 13-13 shown in FIG. 12, the exemplary valve being in an open position;

FIG. 14 depicts a view of an exemplary piece of plastic tubing suitable for use with the urine bag of the present invention;

FIGS. 15A-15C depict view of an exemplary series of steps in a method of using the urine bag of the present invention;

FIG. 16 depicts a cross-sectional view of the exemplary urine bag shown in FIG. 2 as viewed along line 10-10 shown in FIG. 2 and as viewed when in use; and

FIGS. 17A-17C depict frontal views of exemplary shapes of a penis-containing chamber of the exemplary urine bag shown in FIG. 2 as viewed in FIG. 2.

### DETAILED DESCRIPTION OF THE INVENTION

The present invention is directed to urine bags. The present invention is further directed to methods of making, as well as methods of using urine bags.

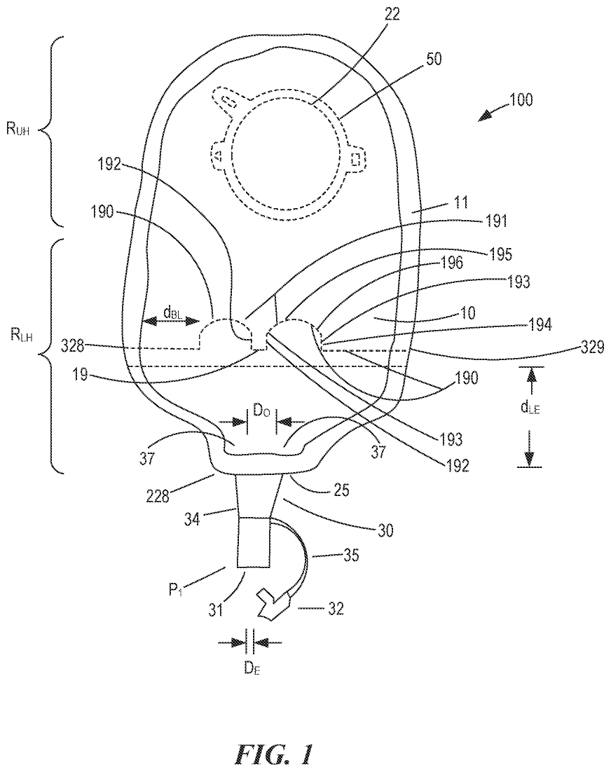

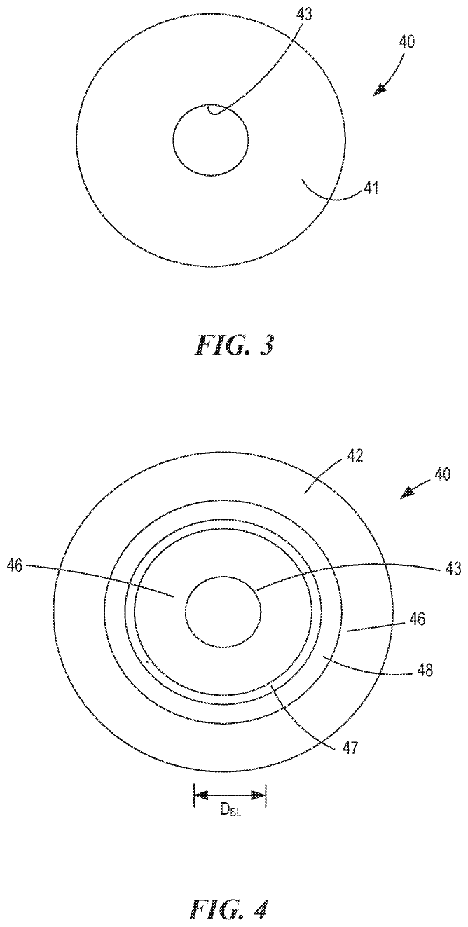

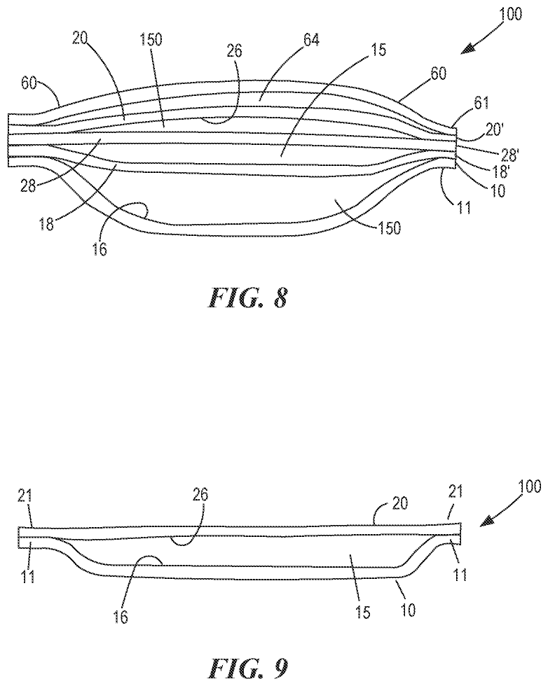

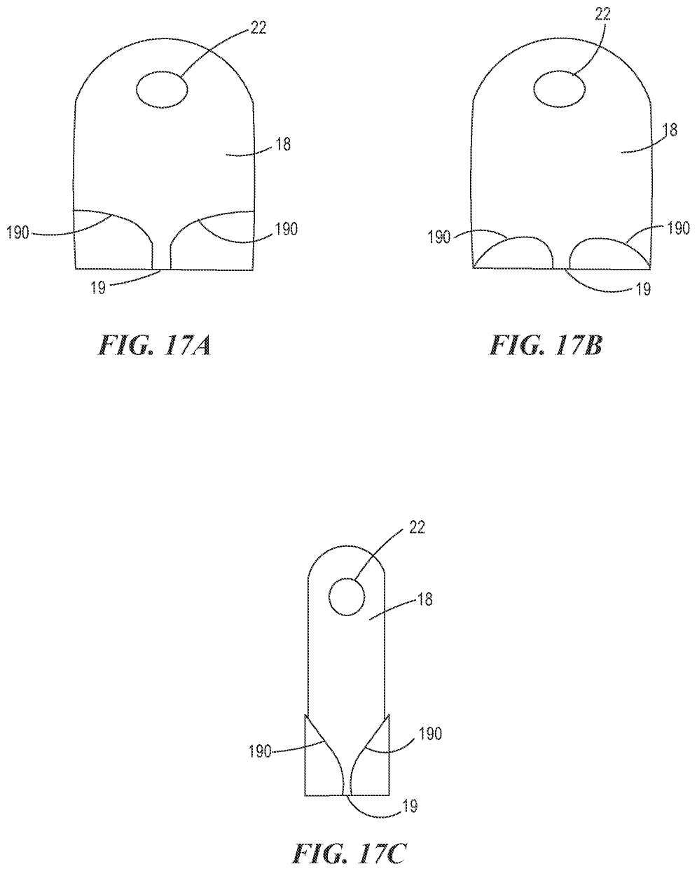

One exemplary urine bag of the present invention is shown in FIGS. 1-17C. As shown in FIGS. 1-2 and 10, exemplary urine bag 100 comprises: front film 10 having front film outer perimeter 11; rear film 20 having rear film outer perimeter 21, the rear film outer perimeter 21 being bonded to the front film outer perimeter 11 so as to form a urine collection chamber 150 between the front film 10 and the rear film 20; inner front film 18 having inner front film outer perimeter 18′; inner rear film 28 having inner rear film outer perimeter 28′, the inner front film outer perimeter 18′ being bonded to the inner rear film outer perimeter 28′ so as to form a penis-containing chamber 15 between the inner front film 18 and the inner rear film 28; an inlet 22 within an upper half R_{UH} of (i) the rear film 20 and (ii) the inner rear film 28 and positioned a distance d_{IE} from the rear film outer perimeter 21, the inlet 22 having an inlet diameter D_{I} and providing access to the penis-containing chamber 15; an outlet 25 extending along a lower edge portion 228 of the front film outer perimeter 11 and the rear film outer perimeter 21, the outlet 25 having an outlet diameter D_{O} and providing an outlet for the urine collection chamber 150; and a fluid flow regulator 19 that provides one-way fluid flow from the penis-containing chamber 15 into the urine collection chamber 150.

As shown in FIGS. 1-2, 7 and 10, exemplary urine bag 100 further comprises: an outlet tubular member 30 extending from the outlet 25 to a position P₁ below the lower edge portion 228, the outlet tubular member 30 providing fluid communication between the urine collection chamber 150 and an exit 31 of the outlet tubular member 30; a stopper 32 sized to fit within the exit 31 of the outlet tubular member 30 so as to prevent fluid flow therethrough; a flange component 50 extending along and connected to an outer perimeter 24 of inlet 22; and a comfort-providing sheet 60 having a comfort-providing sheet outer perimeter 61 bonded to at least a portion 27 of the rear film outer perimeter 21 so as to form an envelope 64 between the comfort-providing sheet 60 and the rear film 20.

As shown in FIGS. 4-6 and 12-14, exemplary urine bag 100 further comprises: an adhesive component 40 that enables the urine bag 100 to be adhesively attached to a user 90 so as to encircle and contain the user's penis 91 within the penis-containing chamber 15; a valve 70 that is connectable to the exit 31 of the outlet tubular member 30, and enables urine sample collection of urine 151 from within the urine collection chamber 150; and a piece of plastic tubing 80 that is connectable to the valve outlet 72.

Further additional embodiments of the present invention are described below. It should be noted that the recitation of numerical ranges by endpoints in any of the embodiments disclosed herein includes all numbers subsumed within that range (e.g., the range of from about 1 to about 5 includes 1, 1.5, 2, 2.75, 3, 3.80, 4, and 5, in increments of 0.0001) and any range within that range (e.g., the range of from about 1.5 to about 3.78 within the range of 1 to 5, in increments of 0.0001).

### ADDITIONAL EMBODIMENTS

Urine Bags:

1. A urine bag 100 comprising:

a front film 10 having a front film outer perimeter 11, and a rear film 20 having a rear film outer perimeter 21, the rear film outer perimeter 21 being bonded to the front film outer perimeter 11 so as to form a urine collection chamber 150 between the front film 10 and the rear film 20;

an inner front film 18 having an inner front film outer perimeter 18′, and an inner rear film 28 having an inner rear film outer perimeter 28′, the inner front film outer perimeter 18′ being bonded to the inner rear film outer perimeter 28′ so as to form a penis-containing chamber 15 between the inner front film 18 and the inner rear film 28;

an inlet 22 within an upper half R_{UH} of (i) the rear film 20 and (ii) the inner rear film 28 and positioned a distance d_{IE} from the rear film outer perimeter 21, the inlet 22 having an inlet diameter D_{I} of at least 20.0 millimeters (mm), said inlet 22 providing access to the penis-containing chamber 15;

an outlet 25 extending along a lower edge portion 228 of the front film outer perimeter 11 and the rear film outer perimeter 21, the outlet 25 having an outlet diameter D_{O} of less than about 20.0 mm, said outlet 25 providing an outlet for the urine collection chamber 150; and

a fluid flow regulator 19 that provides one-way fluid flow from the penis-containing chamber 15 into the urine collection chamber 150.

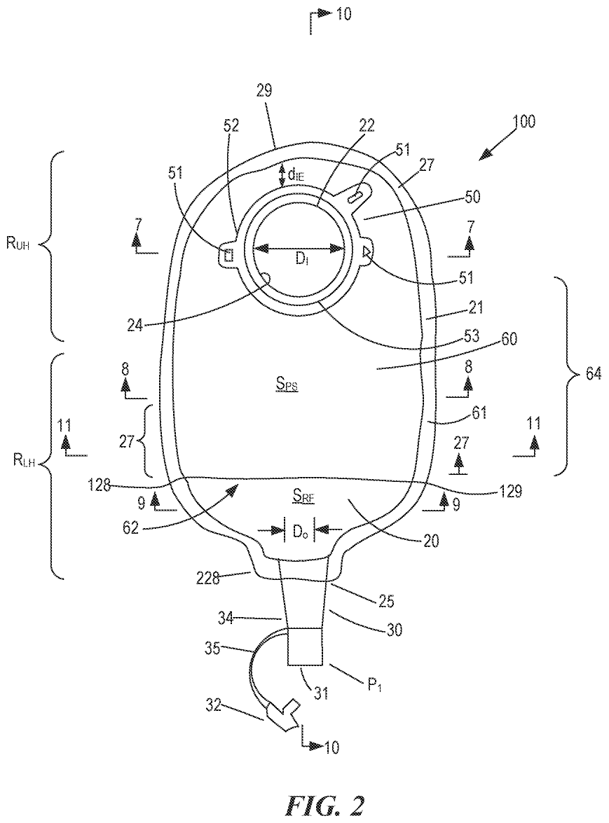

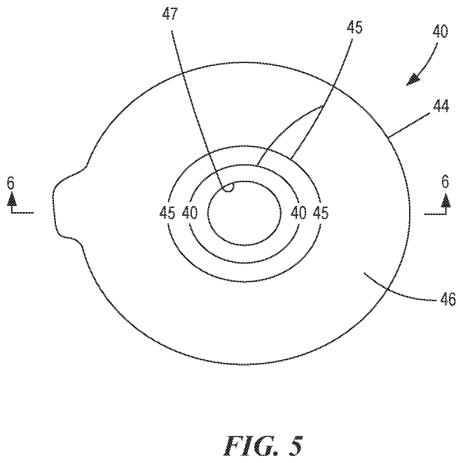

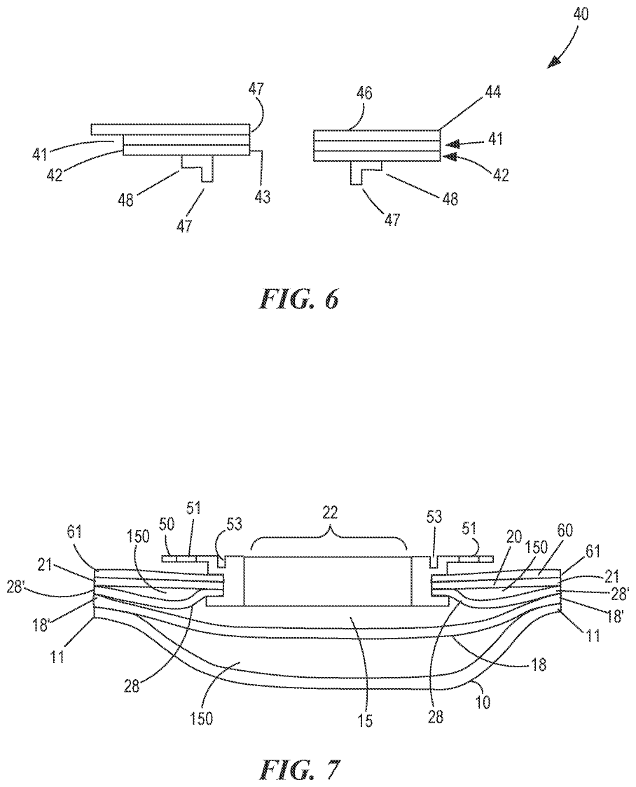

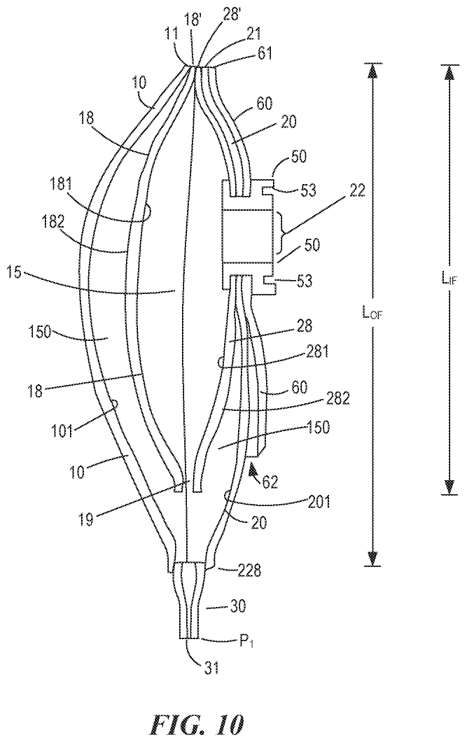

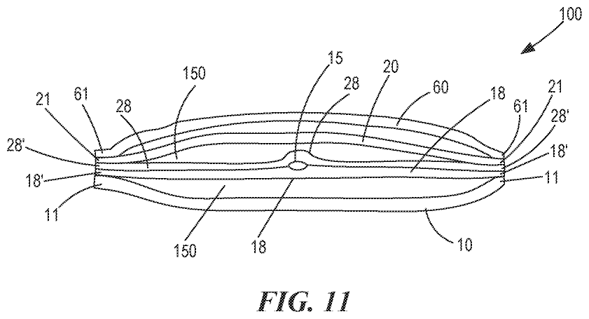

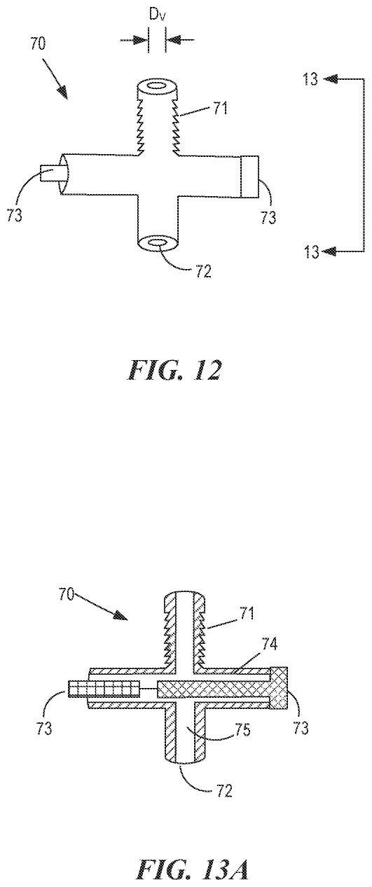

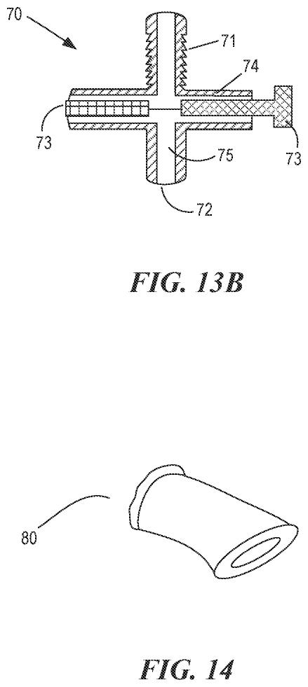

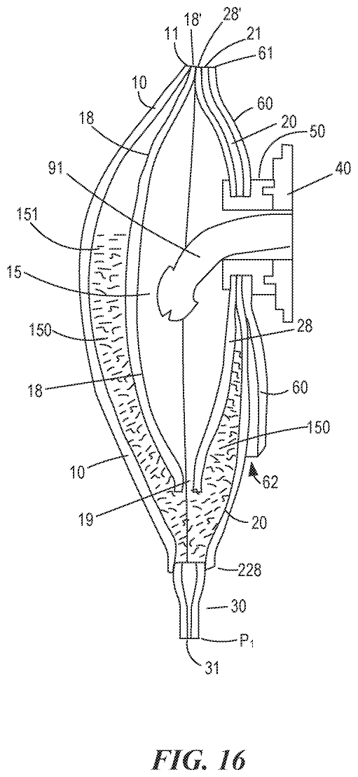

2. The urine bag 100 of embodiment 1, further comprising: an outlet tubular member 30 extending from the outlet 25 to a position P₁ below the lower edge portion 228, the outlet tubular member 30 providing fluid communication between the urine collection chamber 150 and an exit 31 of the outlet tubular member 30.
3. The urine bag 100 of embodiment 1 or 2, further comprising: an adhesive component 40 that enables the urine bag 100 to be adhesively attached to a user 90 so as to encircle and contain the user's penis 91 within the penis-containing chamber 15. See, for example, FIG. 16.
4. The urine bag 100 of any one of embodiments 1 to 3, wherein each of the front film 10, the rear film 20, the inner front film 18, and the inner rear film 28 is independently transparent, translucent, or nontransparent.
5. The urine bag 100 of any one of embodiments 1 to 4, wherein the front film 10 is transparent.
6. The urine bag 100 of any one of embodiments 1 to 5, wherein the rear film 20 is transparent.
7. The urine bag 100 of any one of embodiments 1 to 6, wherein the inner front film 18 is transparent.
8. The urine bag 100 of any one of embodiments 1 to 7, wherein the inner rear film 28 is transparent.
9. The urine bag 100 of any one of embodiments 1 to 8, further comprising a film bonding component (not shown) used to bond (i) the rear film outer perimeter 21 to the front film outer perimeter 11, (ii) the inner front film outer perimeter 18′ to the inner rear film outer perimeter 28′, or (iii) both (i) and (ii). Suitable film bonding components, when used, comprise a film adhesive, a thermoformable film or tape, etc. Typically, a film bonding component is not needed when forming bonds between the film layers (i.e., the film material, when heated, forms an adhesive bond with adjacent film layers).
10. The urine bag 100 of any one of embodiments 1 to 9, wherein the urine bag 100 has a substantially oval shape (i.e., the front film outer perimeter 11 and the rear film outer perimeter 21 are bonded to one another so as to form a substantially oval shape).
11. The urine bag 100 of any one of embodiments 1 to 10, wherein the penis-containing chamber 15 is bound by (i) an inner surface 181 of the inner front film 18, (ii) an inner surface 281 of the inner rear film 28, and (iii) inlet 22. See, for example, FIG. 10.
12. The urine bag 100 of any one of embodiments 1 to 11, wherein the urine collection chamber 150 is bound by (i) an inner surface 101 of the front film 10, (ii) an inner surface 201 of the rear film 20, (iii) the outlet 25, and at least one (iv)(a) an outer surface 182 of the inner front film 18, and (iv)(b) an outer surface 282 of the inner rear film 28. See again, for example, FIG. 10.
13. The urine bag 100 of any one of embodiments 1 to 12, wherein the urine collection chamber 150 is bound by (i) an inner surface 101 of the front film 10, (ii) an inner surface 201 of the rear film 20, (iii) the outlet 25, (iv) an outer surface 182 of the inner front film 18, and (v) an outer surface 282 of the inner rear film 28.
14. The urine bag 100 of any one of embodiments 1 to 13, wherein each of the front film 10, the rear film 20, the inner front film 18, and the inner rear film 28 independently has an overall film surface area, and the overall film surface area of each of the front film 10 and the rear film 20 is greater than the overall film surface area of each of the inner front film 18 and the inner rear film 28.
15. The urine bag 100 of any one of embodiments 1 to 14, wherein a length L_{IF} of each of the inner front film 18 and the inner rear film 28 is less than a length L_{OF} of each of the front film 10 and the rear film 20. See again, for example, FIG. 10.
16. The urine bag 100 of any one of embodiments 1 to 15, wherein a width of at least a portion of each of the inner front film 18 and the inner rear film 28 is less than or equal to a width of each of the front film 10 and the rear film 20.
17. The urine bag 100 of any one of embodiments 1 to 16, wherein a width of at least a portion of each of the inner front film 18 and the inner rear film 28 is less than a width of each of the front film 10 and the rear film 20.
18. The urine bag 100 of any one of embodiments 1 to 17, wherein the penis-containing chamber 15 ranges from about 100 cubic centimeters (cm³) to about 800 cm³ (or any value between 100 cm³ and 800 cm³ in increments of 0.1 cm³, e.g., 423.3 cm³, or any range of values between 100 cm³ and 800 cm³ in increments of 0.1 cm³, e.g., from about 230.8 cm³ to about 344.4 cm³).
19. The urine bag 100 of any one of embodiments 1 to 18, wherein the urine collection chamber 150 is capable of holding up to about 750.0 milliliters (ml) of urine 151 (or any value up to about 750.0 ml in increments of 0.1 ml, e.g., 601.3 ml, or any range of values up to about 750.0 ml in increments of 0.1 ml, e.g., from about 430.8 ml to about 621.4 ml).
20. The urine bag 100 of any one of embodiments 1 to 19, wherein the urine collection chamber 150 is capable of holding from about 400.0 g to 600.0 ml of urine 151.
21. The urine bag 100 of any one of embodiments 1 to 20, wherein the urine collection chamber 150 at least partially surrounds the penis-containing chamber 15.
22. The urine bag 100 of any one of embodiments 1 to 21, wherein the urine collection chamber 150 completely surrounds the penis-containing chamber 15.
23. The urine bag 100 of any one of embodiments 1 to 22, wherein the inlet 22 has a circular shape.
24. The urine bag 100 of any one of embodiments 1 to 23, wherein the inlet diameter D_{I} ranges from about 40.0 mm to about 60.0 mm.
25. The urine bag 100 of any one of embodiments 1 to 24, wherein the outlet 25 has a circular shape.
26. The urine bag 100 of any one of embodiments 1 to 25, wherein the outlet diameter D_{O} ranges from about 3.0 mm to about 15.0 mm.
27. The urine bag 100 of any one of embodiments 2 to 26, wherein the exit 31 of the outlet tubular member 30 has an exit diameter DE ranging from about 3.0 mm to about 7.0 mm.
28. The urine bag 100 of any one of embodiments 2 to 27, wherein the outlet tubular member 30 further comprises a stopper 32 sized to fit within the exit 31 of the outlet tubular member 30 so as to prevent fluid flow therethrough.
29. The urine bag 100 of embodiment 28, wherein the stopper 32 is integrally attached to the outlet tubular member 30. For example, as shown in FIGS. 1-2, exemplary stopper 32 is connected to the outlet tubular member 30 along an outer surface 34 of the outlet tubular member 30 via a stopper connector 35.
30. The urine bag 100 of any one of embodiments 1 to 29, wherein the fluid flow regulator 19 comprises an opening 19 formed between the inner front film 18 and the inner rear film 28 along a lower edge of the inner front film 18 and a lower edge of the inner rear film 28.
31. The urine bag 100 of any one of embodiments 1 to 30, wherein the fluid flow regulator 19 comprises a channel 19 formed between the inner front film 18 and the inner rear film 28 along a lower edge of the inner front film 18 and a lower edge of the inner rear film 28.
32. The urine bag 100 of any one of embodiments 1 to 31, wherein the fluid flow regulator 19 comprises an anti-reflux valve 19. Suitable anti-reflux valves are known in the art, such as anti-reflux valves commercially available from Cardinal Healthcare. As urine 151 fills out urine collection chamber 150, the urine 151 exerts tension on a lower edge of the penis-containing chamber 15, preventing urine 151 reflux into the penis-containing chamber 15.
33. The urine bag 100 of any one of embodiments 1 to 32, further comprising a flange component 50 extending along and connected to an outer perimeter 24 of said inlet 22.
34. The urine bag 100 of embodiment 33, wherein the flange component 50 comprising one or more flange holes 51 used to attach to a suspender to alleviate traction on the skin by adhesive positioned along a flange periphery 52 of the flange component 50.
35. The urine bag 100 of any one of embodiments 1 to 34, further comprising a comfort-providing sheet 60 having a comfort-providing sheet outer perimeter 61, the comfort-providing sheet outer perimeter 61 being bonded to at least a portion 27 of the rear film outer perimeter 21 so as to form an envelope 64 between the comfort-providing sheet 60 and the rear film 20. See, for example, FIG. 2.
36. The urine bag 100 of embodiment 35, wherein the comfort-providing sheet 60 comprises a perforated sheet 60.
37. The urine bag 100 of embodiment 35 or 36, wherein the comfort-providing sheet 60 comprises a nonwoven fabric that provides comfortable contact with the skin (e.g., a spunbonded fabric, a meltblown fabric, a stitch-bonded fabric, a spunlaced fabric, etc.).
38. The urine bag 100 of any one of embodiments 35 to 37, wherein the comfort-providing sheet 60 is bonded to the rear film 20 (i) along the rear film outer perimeter 21 extending from a lower left-side point 128 along upper edge 29 to a lower right-side point 129 and (ii) along the inlet 22.
39. The urine bag 100 of any one of embodiments 35 to 38, wherein the envelope 64 between the comfort-providing sheet 60 and the rear film 20 comprises an envelope opening 62, said envelope opening 62 being along a lower half R_{LH} of the rear film 20.
40. The urine bag 100 of embodiment 39, wherein the envelope opening 62 extends substantially horizontally across the rear film 20. See again, for example, FIG. 2.
41. The urine bag 100 of any one of embodiments 35 to 40, wherein the comfort-providing sheet 60 has a sheet surface area S_{PS} that is less than a rear film surface area S_{RF} of the rear film 20.
42. The urine bag 100 of any one of embodiments 35 to 41, wherein the flange component 50 is bonded to the comfort-providing sheet 60, the rear film 20, and the inner rear film 28. In other embodiments, the flange component 50 may be bonded to only the rear film 20, and the inner rear film 28.
43. The urine bag 100 of any one of embodiments 33 to 42, wherein the flange component 50 further comprises a flange connector 53 designed to connect to the adhesive component 40.
44. The urine bag 100 of any one of embodiments 3 to 43, wherein the adhesive component 40 comprises a pressure-sensitive adhesive 41.
45. The urine bag 100 of embodiment 44, wherein the adhesive component 40 comprises a backing layer 42 that supports the pressure-sensitive adhesive 41, said backing layer 42 having a backing layer opening 43 therethrough. See, for example, exemplary backing layer 42 shown in FIG. 6. It should be noted that although exemplary backing layer 42 is shown as being smaller than removable release liner 44, exemplary backing layer 42 may be larger than removable release liner 44, and have any desired shape (e.g., an overall circular shape, square shape, rectangular shape, etc.).
46. The urine bag 100 of embodiment 45, wherein the backing layer opening 43 has an opening diameter D_{BL} of at least 20.0 mm. Typically, opening diameter D_{BL} ranges from about 40.0 mm to about 60.0 mm.
47. The urine bag 100 of any one of embodiments 44 to 46, wherein the adhesive component 40 further comprising a removable release liner 44 positioned over the pressure-sensitive adhesive 41.
48. The urine bag 100 of embodiment 47, wherein the removable release liner 44 comprises one or more concentric circle cut lines 45 printed along a removable release liner outer surface 46 opposite the pressure-sensitive adhesive 41. As shown in FIG. 5, exemplary removable release liner 44 may further comprise a release liner hole 47 within the removable release liner 44. When present the release liner hole 47 may be smaller or the same size as the backing layer opening 43.
49. The urine bag 100 of embodiment 48, wherein the one or more concentric circle cut lines 45 comprise at least one of: (i) a 40 mm cut line 45, (ii) a 45 mm cut line 45, (iii) a 50 mm cut line 45, and (iv) a 55 mm cut line 45.
50. The urine bag 100 of any one of embodiments 45 to 49, wherein the backing layer 42 further comprises a backing layer connector 48 positioned opposite the pressure-sensitive adhesive 41, said backing layer connector 48 at least partially encircling the backing layer opening 43.
51. The urine bag 100 of embodiment 50, wherein the backing layer connector 48 completely encircles the backing layer opening 43.
52. The urine bag 100 of embodiment 50 or 51, wherein the backing layer connector 46 is sized and designed to connect to the flange connector 53 of the flange component 50. In some embodiments, the backing layer connector 46 comprises a backing layer male connecting component 47 that engages with the flange connector 53, wherein the flange connector 53 comprises a female connecting component 53. See, for example, FIGS. 6-7 which depict a backing layer male connecting component 47 and a female connecting component 53. In other embodiments (not shown), the backing layer connector 46 comprises a backing layer female connecting component 47 that engages with the flange connector 53, wherein the flange connector 53 comprises a male connecting component 53.
53. The urine bag 100 of any one of embodiments 50 to 52, wherein the backing layer connector 46 is connected to the flange connector 53 of the flange component 50.
54. The urine bag 100 of any one of embodiments 1 to 53, further comprising a valve 70 that is connectable to the exit 31 of the outlet tubular member 30, wherein the valve 70 enables urine sample collection of urine 151 from within the urine collection chamber 150. See, for example, FIGS. 12-13B.
55. The urine bag 100 of embodiment 54, wherein the valve 70 comprises a valve connector 71 sized to fit within the exit 31 of the outlet tubular member 30, and a valve outlet 72 opposite the valve connector 71.
56. The urine bag 100 of embodiment 54 or 55, wherein the valve 70 comprises a valve plunger 73 that is movable from a closed position (i.e., see FIG. 13A) to an open position (i.e., see FIG. 13B) and vice versa. As shown in the cross-sectional view of FIGS. 13A-13B, valve plunger 73 moves within a valve channel 74 that is 90° oriented with a valve channel 75.
57. The urine bag 100 of any one of embodiments 54 to 56, wherein the valve 70 has a T-shape. As shown in FIGS. 12-13B, exemplary valve 70 comprises a vertical portion of the T-shape (i.e., the portion in which valve channel 75 extends) and a horizontal portion of the T-shape, namely, valve channel 74 (i.e., the portion in which valve plunger 73 moves).
58. The urine bag 100 of any one of embodiments 54 to 57, wherein the valve 70 has a valve inner diameter D_{V} ranging from about 3.0 mm to about 7.0 mm.
59. The urine bag 100 of any one of embodiments 55 to 58, further comprising a piece of plastic tubing 80 that is connectable to the valve outlet 72. As shown in FIG. 14, exemplary plastic tubing 80 comprises tubing end 81 that is sized to fit over the valve outlet 72.
60. The urine bag 100 of any one of embodiments 1 to 59, wherein the front film 10 is bonded to the rear film 20 along a complete length of the rear film outer perimeter 21 except for the lower edge portion 228 in which the outlet tubular member 30 is positioned.
61. The urine bag 100 of any one of embodiments 1 to 60, wherein the inner front film 18 is bonded to the inner rear film 28 along a complete length of (i) the inner front film outer perimeter 18′ and (ii) the inner rear film outer perimeter 28′ except for the lower edge portion 228 in which the fluid flow regulator 19 is positioned.
62. The urine bag 100 of any one of embodiments 1 to 61, wherein the front film 10 is bonded to (i) the rear film 20, (ii) the inner front film 18, and (iv) the inner rear film 28 along a length of (a) the front film outer perimeter 11, (b) the rear film outer perimeter 21, (c) the inner front film outer perimeter 18′ and (d) the inner rear film outer perimeter 28′ that extends from (1) a left-hand side location 328 positioned along the front film outer perimeter 11 and a distance d_{LE} above the lower edge portion 228, along an upper edge of the rear film 20, to a right-hand side location 329 positioned along the front film outer perimeter 11 and the distance d_{LE} above the lower edge portion 228. See, for example, FIG. 1.
63. The urine bag 100 of any one of embodiments 35 to 62, wherein the front film 10 is bonded to (i) the rear film 20, (ii) the inner front film 18, (iv) the inner rear film 28, and (v) the comfort-providing sheet 60 along a length of (a) the front film outer perimeter 11, (b) the rear film outer perimeter 21, (c) the inner front film outer perimeter 18′, (d) the inner rear film outer perimeter 28′, and (e) the comfort-providing sheet outer perimeter 61 that extends from (1) a left-hand side location 328 positioned along the front film outer perimeter 11 and a distance d_{LE} above the lower edge portion 228, along an upper edge of the rear film 20, to a right-hand side location 329 positioned along the front film outer perimeter 11 and the distance d_{LE} above the lower edge portion 228. It should be noted that the comfort-providing sheet 60 may be bonded to the rear film 20 along any length of the comfort-providing sheet outer perimeter 61 and the rear film outer perimeter 21 with the bond being a single continuous bond or multiple separate bonds.
64. The urine bag 100 of embodiment 63, wherein (i) the inner front film 18 and (ii) the inner rear film 28 are bonded to one another along one or more bond locations 190 that extend a distance d_{BL} into the (i) the inner front film 18 and (ii) the inner rear film 28 from the inner front film outer perimeter 18′ and the inner rear film outer perimeter 28′.
65. The urine bag 100 of embodiment 64, wherein the one or more bond locations 190 comprise two bond locations 190 within a lower half R_{LH} of (i) the inner front film 18 and (ii) the inner rear film 28.
66. The urine bag 100 of embodiment 64 or 65, wherein each of the one or more bond locations 190 is positioned a distance d_{LE} above the lower edge portion 228.
67. The urine bag 100 of any one of embodiments 64 to 66, wherein each of the one or more bond locations 190 comprises a bond line 191 at least a portion 192 of which extends vertically along inner surfaces 181 and 281 of the inner front film 18 and the inner rear film 28, respectively.
68. The urine bag 100 of any one of embodiments 64 to 67, wherein each of the one or more bond locations 190 comprises a bond line 191 at least a portion 193 of which extends horizontally along inner surfaces 181 and 281 of the inner front film 18 and the inner rear film 28, respectively.
69. The urine bag 100 of any one of embodiments 64 to 68, wherein each of the one or more bond locations 190 comprises a bond line 191 comprising (i) a first vertically-extending portion 192 positioned above an outer edge 37 of the outlet tubular member 30, (ii) an angularly-extending portion 193 that extends from an upper end 195 of the first vertically-extending portion 192 towards the inner front film outer perimeter 18′ of the inner front film 18 and the inner rear film outer perimeter 28′ of the inner rear film 28, and (iii) a second vertically-extending portion 194 that extends downward from an outer edge 196 of the angularly-extending portion 194 so as to be substantially parallel with the first vertically-extending portion 192. See, for example, exemplary bond lines 190 shown in FIG. 1.

Kits Comprising a Urine Bag:

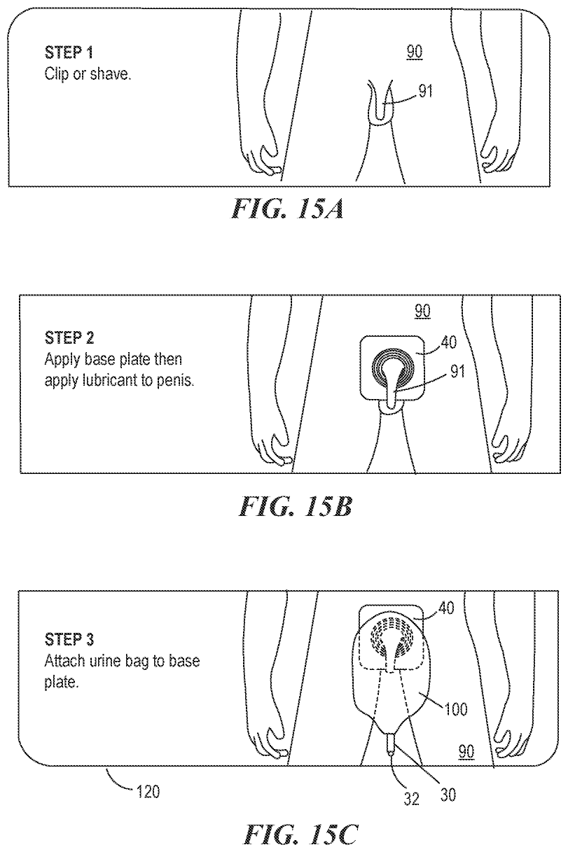

70. A kit comprising the urine bag 100 of any one of embodiments 1 to 69 and one or more additional kit components, wherein said one or more additional kit components comprise (i) a container of lubricating jelly (not shown), (ii) an instruction sheet 120 describing how to use the urine bag (see, FIGS. 15A-15B), (iii) any combination of (i) to (ii).

Methods of Making Urine Bags:

71. A method of making the urine bag 100 of any one of embodiments 1 to 69, said method comprising: bonding the front film 10 having the front film outer perimeter 11 to the rear film 20 having the rear film outer perimeter 21 so as to form the urine collection chamber 150 between the front film 10 and the rear film 20; bonding the inner front film 18 having the inner front film outer perimeter 18′ to the inner rear film 28 having the inner rear film outer perimeter 28′ so as to form the penis-containing chamber 15 between the inner front film 18 and the inner rear film 28; forming the inlet 22 within an upper half R_{UH} of (i) the rear film 20 and (ii) the inner rear film 28 and positioned a distance d_{IE} from the rear film outer perimeter 21, the inlet 22 having an inlet diameter D_{I} of at least 20.0 mm, and providing access to the penis-containing chamber 15; forming the outlet 25 extending along a lower edge portion 228 of the front film outer perimeter 11 and the rear film outer perimeter 21, the outlet 25 having an outlet diameter D_{O} of less than about 20.0 mm, and providing an outlet for the urine collection chamber 150; and positioning a fluid flow regulator 19 between the penis-containing chamber 15 and the urine collection chamber 150 so as to provide one-way fluid flow from the penis-containing chamber 15 into the urine collection chamber 150. In some embodiments, the bonding steps and the positioning steps of the above embodiment are performed simultaneously. Any method may be used to bond layers to one another. Suitable methods include, but are not limited to, thermally bonding, adhesive bonding, etc.
72. The method of embodiment 71, wherein said positioning step comprises: bonding a lower edge portion of the inner front film 18 to a lower edge portion of the inner rear film 28.
73. The method of embodiment 71 or 72, wherein said positioning step comprises: bonding a lower edge portion of the inner front film 18 to a lower edge portion of the inner rear film 28 so as to form an opening 19 along the lower edge of the inner front film 18 and the lower edge of the inner rear film 28.
74. The method of any one of embodiments 71 to 73, wherein said positioning step comprises: bonding a lower edge portion of the inner front film 18 to a lower edge portion of the inner rear film 28 so as to form a channel 19 formed between the inner front film 18 and the inner rear film 28 along the lower edge of the inner front film 18 and the lower edge of the inner rear film 28.
75. The method of any one of embodiments 71 to 74, wherein said forming the inlet 22 step comprises: a cutting step or a stamping step.
76. The method of any one of embodiments 71 to 75, further comprising: connecting the outlet tubular member 30 to the front film 10 and the rear film 20.
77. The method of any one of embodiments 71 to 76, further comprising: cutting the front film 10, the rear film 20, the inner front film 18, and the inner rear film 28 from one or more film sheets (not shown).
78. The method of any one of embodiments 71 to 77, further comprising: forming the stopper 32 that is sized to fit within the exit 31 of the outlet tubular member 30 so as to prevent fluid flow therethrough.
79. The method of embodiment 78, wherein said forming the stopper 32 step comprises integrally molding the stopper 32 so as to be connected to the outlet tubular member 30. As shown in FIG. 1, in some embodiments, the stopper 32 is integrally connected to the outlet tubular member 30 via a stopper connector 35. Any method may be used to form the stopper 32. Suitable methods include, but are not limited to, molding, injection molding, 3D printing, etc.
80. The method of any one of embodiments 71 to 79, further comprising: forming the flange component 50.
81. The method of embodiment 80, wherein said forming the flange component 50 step comprises molding the flange component 50. Any method may be used to form the flange component 50. Suitable methods include, but are not limited to, molding, injection molding, 3D printing, etc.
82. The method of any one of embodiments 71 to 81, further comprising: attaching the flange component 50 to the rear film 20. Any method may be used to connect the flange component 50 to the rear film 20. Suitable methods include, but are not limited to, thermally bonding, adhesive bonding, etc.
83. The method of any one of embodiments 71 to 82, further comprising: bonding the comfort-providing sheet 60 to the rear film 20 so that the comfort-providing sheet outer perimeter 61 is bonded to the rear film outer perimeter 21. Any method may be used to bond the comfort-providing sheet 60 to the rear film 20. Suitable methods include, but are not limited to, thermally bonding, adhesive bonding, etc.
84. The method of any one of embodiments 71 to 83, further comprising: cutting the comfort-providing sheet 60 from one or more sheets of material (not shown).
85. The method of any one of embodiments 71 to 84, further comprising: attaching the flange component 50 to the comfort-providing sheet 60, the rear film 20, and the inner rear film 28. Any method may be used to bond the flange component 50 to the comfort-providing sheet 60, the rear film 20, and the inner rear film 28. Suitable methods include, but are not limited to, thermally bonding, adhesive bonding, etc. As noted above, in some embodiments, the flange component 50 is attached to only the to the rear film 20, and the inner rear film 28.
86. The method of any one of embodiments 71 to 85, further comprising: applying the pressure-sensitive adhesive 41 onto the backing layer 42. Any method may be used to apply the pressure-sensitive adhesive 41 onto the backing layer 42. Suitable methods include, but are not limited to, a coating step, a spray-coating step, a dip coating step, a lamination step, etc.
87. The method of any one of embodiments 71 to 86, further comprising: cutting the backing layer opening 43 within the backing layer 42.
88. The method of any one of embodiments 71 to 87, further comprising: positioning the removable release liner 44 over the pressure-sensitive adhesive 41.
89. The method of any one of embodiments 71 to 88, further comprising: printing the one or more concentric circle cut lines 45 along the removable release liner outer surface 46 opposite the pressure-sensitive adhesive 41.
90. The method of any one of embodiments 71 to 89, further comprising: forming the backing layer connector 48.
91. The method of embodiment 90, wherein said forming step comprises molding the backing layer connector 48. Any method may be used to form the backing layer connector 48. Suitable methods include, but are not limited to, molding, injection molding, 3D printing, etc.
92. The method of any one of embodiments 71 to 91, further comprising: attaching the backing layer connector 48 to the backing layer 42. Any method may be used to attach the backing layer connector 48 to the backing layer 42. Suitable methods include, but are not limited to, thermally bonding, adhesive bonding, etc.
93. The method of any one of embodiments 71 to 92, further comprising: forming the valve 70.
94. The method of embodiment 93, wherein said forming step comprises at least one molding step. Any method may be used to form the valve 70 (and its individual components). Suitable methods include, but are not limited to, molding, injection molding, 3D printing, etc.
95. The method of any one of embodiments 71 to 94, wherein said bonding step of bonding the inner front film 18 to the inner rear film 28 further comprises forming one or more bond locations 19 that extend a distance d_{BL} into the (i) the inner front film 18 and (ii) the inner rear film 28 from the inner front film outer perimeter 18′ and the inner rear film outer perimeter 28′. Any method may be used to form the one or more bond locations 19. Suitable methods include, but are not limited to, thermally bonding, adhesive bonding, etc.

Methods of Using Urine Bags:

96. A method of using the urine bag 100 of any one of embodiments 1 to 69, said method comprising: adhesively attaching the urine bag 100 to the user 90 so as to encircle and contain the user's penis 91 within the penis-containing chamber 15; and collecting urine 151 from the user 90 within the urine collection chamber 150. See, for example, the method steps shown in FIGS. 15A-15C.
97. The method of embodiment 96, wherein said adhesively attaching step comprises adhesively attaching the adhesive component 40 to the user 90. See, for example, FIG. 15B.
98. The method of embodiment 96 or 97, wherein said adhesively attaching step comprises adhesively attaching the adhesive component 40 to the user 90, the adhesive component 40 comprising the backing layer 42 with pressure-sensitive adhesive 41 thereon and the backing layer connector 48 opposite the pressure-sensitive adhesive 41, and connecting the flange component 50 to the backing layer 42 via the backing layer connector 48 and the flange connector 53. See, for example, FIG. 15C.
99. The method of any one of embodiments 96 to 98, further comprising: inserting the valve connector 72 of the valve 70 into the exit 31 of the outlet tubular member 30.
100. The method of any one of embodiments 96 to 99, wherein said collecting step comprises collecting urine 151 over a 48-hour (hr) period.
101. The method of any one of embodiments 96 to 100, further comprising: opening the valve 70 to take a urine sample from the urine collection chamber 150; and closing the valve 70.
102. The method of embodiment 101, further comprising: repeating said opening and closing steps numerous times over a 48 hr period.

It should be understood that although the above-described urine bags, and methods are described as “comprising” one or more components or steps, the above-described urine bags, kits, and methods may “comprise,” “consists of,” or “consist essentially of” any of the above-described components or steps of the urine bags, kits, and methods. Consequently, where the present invention, or a portion thereof, has been described with an open-ended term such as “comprising,” it should be readily understood that (unless otherwise stated) the description of the present invention, or the portion thereof, should also be interpreted to describe the present invention, or a portion thereof, using the terms “consisting essentially of” or “consisting of” or variations thereof as discussed below.

As used herein, the terms “comprises,” “comprising,” “includes,” “including,” “has,” “having,” “contains”, “containing,” “characterized by” or any other variation thereof, are intended to encompass a non-exclusive inclusion, subject to any limitation explicitly indicated otherwise, of the recited components. For example, a urine bag, a kit, and/or a method that “comprises” a list of elements (e.g., components or steps) is not necessarily limited to only those elements (or components or steps), but may include other elements (or components or steps) not expressly listed or inherent to the urine bag, the kit, and/or method.

As used herein, the transitional phrases “consists of” and “consisting of” exclude any element, step, or component not specified. For example, “consists of” or “consisting of” used in a claim would limit the claim to the components, materials or steps specifically recited in the claim except for impurities ordinarily associated therewith (i.e., impurities within a given component). When the phrase “consists of” or “consisting of” appears in a clause of the body of a claim, rather than immediately following the preamble, the phrase “consists of” or “consisting of” limits only the elements (or components or steps) set forth in that clause; other elements (or components) are not excluded from the claim as a whole.

As used herein, the transitional phrases “consists essentially of” and “consisting essentially of” are used to define urine bags, and methods that include materials, steps, features, components, or elements, in addition to those literally disclosed, provided that these additional materials, steps, features, components, or elements do not materially affect the basic and novel characteristic(s) of the claimed invention. The term “consisting essentially of” occupies a middle ground between “comprising” and “consisting of”.

Further, it should be understood that the herein-described urine bags, kits, and methods may comprise, consist essentially of, or consist of any of the herein-described components and features, as shown in the figures with or without any feature(s) not shown in the figures. In other words, in some embodiments, the urine bags, kits, and/or methods of the present invention do not have any additional features other than those shown in the figures, and such additional features, not shown in the figures, are specifically excluded from the urine bags, kits, and/or methods. In other embodiments, the urine bags, kits, and/or methods of the present invention do have one or more additional features that are not shown in the figures.

The present invention is described above and further illustrated below by way of examples, which are not to be construed in any way as imposing limitations upon the scope of the invention. On the contrary, it is to be clearly understood that resort may be had to various other embodiments, modifications, and equivalents thereof which, after reading the description herein, may suggest themselves to those skilled in the art without departing from the spirit of the present invention and/or the scope of the appended claims.

### Example 1

### Preparation of Urine Bags

Exemplary urine bags as shown and described in FIGS. 1-17C were prepared and used as shown and discussed herein.

While the specification has been described in detail with respect to specific embodiments thereof, it will be appreciated that those skilled in the art, upon attaining an understanding of the foregoing, may readily conceive of alterations to, variations of, and equivalents to these embodiments. Accordingly, the scope of the present invention should be assessed as that of the appended claims and any equivalents thereto.

What is claimed is:

1. A urine bag comprising:
a front film having a front film outer perimeter, and a rear film having a rear film outer perimeter, the rear film outer perimeter being bonded to the front film outer perimeter so as to form a urine collection chamber between the front film and the rear film;
an inner front film having an inner front film outer perimeter, and an inner rear film having an inner rear film outer perimeter, the inner front film outer perimeter being bonded to the inner rear film outer perimeter so as to form a penis-containing chamber between the inner front film and the inner rear film;
an inlet within an upper half R_{UH} of (i) the rear film and (ii) the inner rear film and positioned a distance d_{IE} from the rear film outer perimeter, the inlet having an inlet diameter D_{I} of at least 20.0 millimeters (mm), said inlet providing access to the penis-containing chamber;
an outlet extending along a lower edge portion of the front film outer perimeter and the rear film outer perimeter, the outlet having an outlet diameter D_{O} of less than about 20.0 mm, said outlet providing an outlet for the urine collection chamber; and
a fluid flow regulator that provides one-way fluid flow from the penis-containing chamber into the urine collection chamber.

2. The urine bag of claim 1, further comprising:
an outlet tubular member extending from the outlet to a position P₁ below the lower edge portion, the outlet tubular member providing fluid communication between the urine collection chamber and an exit of the outlet tubular member.

3. The urine bag of claim 2, further comprising: an adhesive component that enables the urine bag to be adhesively attached to a user so as to encircle and contain the user's penis within the penis-containing chamber.

4. The urine bag of claim 1, wherein the penis-containing chamber is bound by (i) an inner surface of the inner front film, (ii) an inner surface of the inner rear film, and (iii) inlet.

5. The urine bag of claim 1, wherein the urine collection chamber is bound by (i) an inner surface of the front film, (ii) an inner surface of the rear film, (iii) the outlet, and at least one (iv)(a) an outer surface of the inner front film, and (iv)(b) an outer surface of the inner rear film.

6. The urine bag of claim 1, wherein the urine collection chamber is bound by (i) an inner surface of the front film, (ii) an inner surface of the rear film, (iii) the outlet, (iv) an outer surface of the inner front film, and (v) an outer surface of the inner rear film.

7. The urine bag of claim 1, wherein (a) the penis-containing chamber ranges from about 100 cubic centimeters (cm³) to about 800 cm³ and, (b) the urine collection chamber is capable of holding up to about 750.0 milliliters (ml) of urine.

8. The urine bag of claim 3, wherein the outlet tubular member further comprises a stopper sized to fit within the exit of the outlet tubular member so as to prevent fluid flow therethrough.

9. The urine bag of claim 1, wherein the fluid flow regulator comprises an opening formed between the inner front film and the inner rear film along a lower edge of the inner front film and a lower edge of the inner rear film.

10. The urine bag of claim 1, wherein the fluid flow regulator comprises a channel formed between the inner front film and the inner rear film along a lower edge of the inner front film and a lower edge of the inner rear film.

11. The urine bag of claim 1, further comprising a comfort-providing sheet having a comfort-providing sheet outer perimeter, the comfort-providing sheet outer perimeter being bonded to at least a portion of the rear film outer perimeter so as to form an envelope between the comfort-providing sheet and the rear film.

12. The urine bag of claim 3, further comprising a valve that is connectable to the exit of the outlet tubular member, wherein the valve enables urine sample collection of urine from within the urine collection chamber.

13. The urine bag of claim 11, wherein the front film is bonded to (i) the rear film, (ii) the inner front film, (iv) the inner rear film, and (v) the comfort-providing sheet along a length of (a) the front film outer perimeter, (b) the rear film outer perimeter, (c) the inner front film outer perimeter, (d) the inner rear film outer perimeter, and (e) the comfort-providing sheet outer perimeter that extends from (1) a left-hand side location positioned along the front film outer perimeter and a distance d_{LE} above the lower edge portion, along an upper edge of the rear film, to a right-hand side location positioned along the front film outer perimeter and the distance d_{LE} above the lower edge portion.

14. The urine bag of claim 13, wherein (i) the inner front film and (ii) the inner rear film are bonded to one another along one or more bond locations that extend a distance d_{BL} into the (i) the inner front film and (ii) the inner rear film from the inner front film outer perimeter and the inner rear film outer perimeter.

15. A kit comprising (a) the urine bag of claim 1, and (b) one or more additional kit components, wherein said one or more additional kit components comprise one or more of: (i) a container of lubricating jelly, and (ii) an instruction sheet describing how to use the urine bag.

16. A method of making the urine bag of claim 1, said method comprising:
bonding the front film having the front film outer perimeter to the rear film having the rear film outer perimeter so as to form the urine collection chamber between the front film and the rear film;
bonding the inner front film having the inner front film outer perimeter to the inner rear film having the inner rear film outer perimeter so as to form the penis-containing chamber between the inner front film and the inner rear film;
forming the inlet within an upper half R_{UH} of (i) the rear film and (ii) the inner rear film and positioned a distance d_{IE} from the rear film outer perimeter, the inlet having an inlet diameter D_{I} of at least 20.0 mm, and providing access to the penis-containing chamber;
forming the outlet extending along a lower edge portion of the front film outer perimeter and the rear film outer perimeter, the outlet having an outlet diameter D_{O} of less than about 20.0 mm, and providing an outlet for the urine collection chamber; and
positioning a fluid flow regulator between the penis-containing chamber and the urine collection chamber so as to provide one-way fluid flow from the penis-containing chamber into the urine collection chamber.

17. The method of claim 16, wherein said positioning step comprises:
bonding a lower edge portion of the inner front film to a lower edge portion of the inner rear film so as to form an opening along the lower edge of the inner front film and the lower edge of the inner rear film.

18. The method of claim 17, wherein said bonding step of bonding the inner front film to the inner rear film further comprises forming one or more bond locations that extend a distance d_{BL} into the (i) the inner front film and (ii) the inner rear film from the inner front film outer perimeter and the inner rear film outer perimeter.

19. A method of using the urine bag of claim 1, said method comprising:
adhesively attaching the urine bag to the user so as to encircle and contain the user's penis within the penis-containing chamber; and
collecting urine from the user within the urine collection chamber.

20. The method of claim 19, wherein said collecting step comprises collecting urine over a 48-hour (hr) period.
